(19) United States
(12) Patent Application Publication
(10) Pub. No.: US 2009/0170967 A1
(43) Pub. Date: Jul. 2, 2009

(54) CONCURRENT OXIDATION AND STEAM METHANE REFORMING PROCESS AND REACTOR THEREFOR

(76) Inventors: Lixin You, Sugar Land, TX (US); Crutis Lee Krause, Houston, TX (US); Kevin Hoa Nguyen, Missouri City, TX (US)

Correspondence Address: THE DOW CHEMICAL COMPANY;JONES DAY, 717 TEXAS, SUITE 3300, HOUSTON, TX, 77002-2712, US

(21) Appl. No.: 11/966,722

(22) Filed: Dec. 28, 2007

(51) Int. Cl. C01B 3/26; C01B 3/02; C07C 27/06; B01J 8/18

(52) U.S. Cl. 518/702; 252/373; 423/648.1; 422/142; 422/146

(57) ABSTRACT

A process for preparation of a synthesis gas and/or hydrogen by concurrently providing an oxidation reactant stream through an oxidation chamber and a reforming reactant stream through a steam, reforming chamber is described. Also provided is a reactor for conducting the reaction.

### FIELD

Provided herein is a process for preparation of synthesis gas by concurrently providing an oxidation reactant stream through an oxidation chamber and a reforming reactant stream through a steam reforming chamber. Also provided is a reactor for conducting the reaction.

### BACKGROUND

Steam methane reforming (SMR) reaction is widely used for synthetic gas and hydrogen production. The SMR reaction is strongly endothermic and requires very high temperatures to obtain high methane conversion rates. The high heat required for the SMR process can often be obtained from oxidation/combustion reactions. The heat exchange between the reactions can be facilitated by various devices, including heat exchange plate fins. Often, this heat exchange is the limiting factor for the steam methane reforming reaction rates and methane conversions. The SMR and oxidation reactions are usually carried out in the presence of a catalyst in counter-current flow. In these reactions, the SMR exit stream is usually coupled with the oxidation inlet stream such that the SMR exit stream has high temperature and high methane conversion. However, the oxidation stream often undergoes homogenous combustion at high hydrogen concentration used in the oxidation reaction. The heat generated in the combustion reaction is often damaging for catalyst, as well as, any structure between heat exchange fins and heat conducting surfaces—especially when the combustion temperature is high. Therefore, typically, a large amount of air is used to control the combustion temperature below the catalyst sintering temperature. Unfortunately, this results in high production costs and low methane conversion rates.

There have been extensive efforts, over a long period of time, aimed at improving the speed and efficiency of the SMR reaction. Despite these attempts, there remains a need for a method and apparatus to carry out the SMR reaction more efficiently and cost effectively.

### SUMMARY OF THE INVENTION

In one embodiment, provided herein is a process for preparing a synthesis gas in a reactor by concurrently providing an oxidation reactant stream through an oxidation chamber and a reforming reactant stream through a steam reforming chamber, wherein the oxidation chamber is adjacent to the reforming chamber, and the oxidation chamber and the reforming chamber are separated by a thermally conductive surface.

The oxidation reactant stream flows into the oxidation chamber through an inlet that is situated adjacent to an inlet for the reforming reactant stream in the reforming reactor. The oxidation and steam reforming reactions are conducted in the presence of oxidation and reforming catalysts, respectively. The oxidation and reforming catalysts can be deposited on a plurality of heat exchange fins to enhance the heat transfer and hence, to increase the SMR reaction and CH₄ conversion rates. These heat exchange fins can provide high density surface areas for both high activity catalyst deposition for process intensification and enhanced heat transfer. In certain embodiments, the heat exchange fins are mounted on the thermally conductive surface.

In one embodiment, provided is a process for generating a synthesis gas, comprising: providing an oxidation reactant stream into an oxidation chamber such that the temperature in the oxidation chamber increases from an inlet of the oxidation chamber to an outlet of the oxidation chamber; concurrently providing a reforming reactant stream in a reforming chamber that is adjacent to the oxidation chamber, wherein the oxidation chamber and the reforming chamber are separated by a thermally conductive wall; transferring at least a part of the heat generated in the oxidation chamber to the reforming chamber; and reacting the reforming reactant stream to generate the synthesis gas.

In certain embodiments, the process further comprises feeding the synthesis gas generated in the process to a membrane separator to produce pure hydrogen. In one aspect, the process further comprises feeding the synthesis gas generated to a membrane separator to produce the synthesis gas with a desired H₂ to CO ratio.

In certain embodiments, the synthesis gas generated is used as a feed gas for a Fischer-Tropsch reactor to produce liquid hydrocarbons. In another aspect, the synthesis gas is useful as a feed to an alcohol synthesis reactor to produce alcohols including methanol, ethanol and higher alcohols.

In one aspect, the synthesis gas produced in the membrane separator is used as a feed to a Fischer-Tropsch reactor to produce liquid hydrocarbons. In another aspect, the synthesis gas produced in the membrane separator is used as a feed to an alcohol synthesis reactor to produce alcohols including methanol, ethanol and higher alcohols.

In another embodiment, provided is a reactor for generating the synthesis gas by the process described herein. The reactor comprises an oxidation chamber and a reforming chamber, wherein the oxidation chamber and the reforming chamber are separated by a thermally conductive surface. The reforming chamber comprises an inlet for a reforming reactant stream, an outlet for the reforming product stream, and a reforming catalyst disposed on a plurality of heat exchange fins. The oxidation chamber comprises an inlet for an oxidation reactant stream, an outlet for the oxidation product stream, and an oxidation catalyst disposed on a plurality of heat exchange fins, such that the inlet of the oxidation chamber is adjacent to the inlet of the reforming chamber. In one embodiment, the heat exchange fins are brazed on the thermally conductive surface.

In certain embodiments, the reactor for generating the synthesis gas is further connected to a pressure swing adsorption unit to produce pure hydrogen. In one embodiment, the reactor for generating the synthesis gas is further connected to a membrane separator to produce pure hydrogen or to adjust the ratio of H₂ to CO to a desirable ratio. In one aspect, the reactor for generating the synthesis gas is further connected to a Fischer-Tropsch reactor to produce liquid hydrocarbons. In another aspect, the reactor for generating the synthesis gas is further connected to an alcohol synthesis reactor to produce alcohols. In yet another aspect, the reactor for generating the synthesis gas is further connected to a membrane separator and a Fischer-Tropsch reactor. In a further aspect, the reactor for generating the synthesis gas is further connected to a membrane separator and an alcohol synthesis reactor.

According to one aspect of the process provided herein, the oxidation and steam methane reforming inlet stream temperatures are lower than the hydrogen self-ignition temperature. Thus, only hydrogen and methane catalyst oxidation takes place in the initial stage of the process. Therefore, the air flow required at this stage is much lower than the air flow required in the countercurrent steam methane reforming reaction. As the reaction progresses, significant amounts of hydrogen and methane are oxidized and the oxidation stream temperatures increase to higher than hydrogen and/or methane self ignition temperatures. At this temperature homogeneous combustion occurs, thereby increasing the oxidation and reforming outlet stream temperatures. The increased temperatures result in increased reforming and oxidation efficiency.

In one aspect, the process provided herein requires reduced air flow thereby reducing the cost associated with higher air power consumption. The higher temperatures at the outlets of the reforming and oxidation chamber result in high methane reforming rates.

In another aspect of the process, the direct coupling of the heat generating oxidation reaction with the endothermic steam reforming reaction in adjacent chambers balances heat transfer between the two reactions and efficiency of the reaction is maximized.

### BRIEF DESCRIPTION OF THE DRAWING FIGURES

FIG. 1 is a schematic illustration of concurrent oxidation and steam methane reforming flow described herein.

FIG. 2 is an exemplary temperature profile for an embodiment of the process described herein.

FIGS. 3a, 3b and 3c depict exemplary heat exchange fins for use in the processes and reactor provided herein.

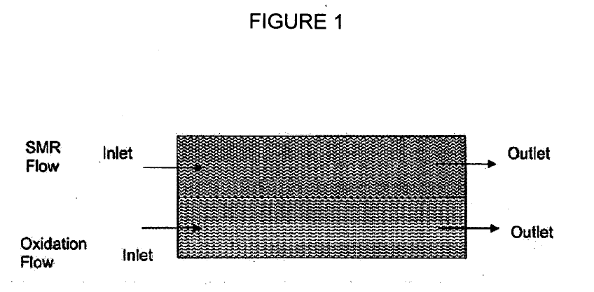

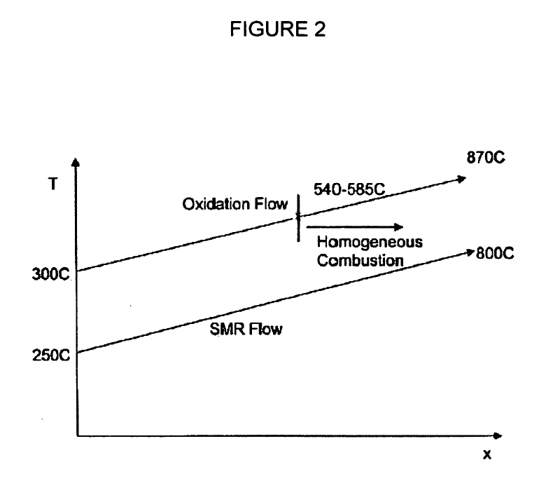

### DEFINITIONS

To facilitate the understanding of the subject matter disclosed herein, a number of terms, abbreviations or other shorthand as used herein are defined below. Any term, abbreviation or shorthand not defined is understood to have the ordinary meaning used by a skilled artisan contemporaneous with the submission of this application.

As used herein, “oxidation stream” refers to a gas stream comprising hydrogen, methane, carbon monoxide and oxygen.

As used herein, “oxygen” or “oxygen source” refers to a gas comprising molecular oxygen, air or other oxidants, such as nitrogen oxides, which can function as a source of oxygen. The oxygen source may be carbon dioxide, carbon monoxide or a peroxide (e.g., hydrogen peroxide). Gaseous mixtures containing oxygen, such as mixtures of oxygen and air, or mixtures of oxygen and an inert gas (e.g., helium, argon, etc.) or a diluent gas (e.g., carbon dioxide, water vapor, etc.) may also be used as oxygen source in the oxidation stream.

As used herein, “reforming reactant stream” refers to a gas stream comprising water and methane.

As used herein, “water” generally includes, liquid water, combinations of liquid water and steam, and steam.

As used herein, “oxidation chamber” refers to a reaction, chamber in the reactor where the oxidation and combustion reactions take place.

As used herein, “reforming chamber” refers to the reaction chamber where the steam methane reforming reaction takes place.

As used herein, “concurrently” or “concurrent” refers to parallel flow of gas streams in the same direction. The gas streams may be provided simultaneously or within a short time interval of each other. For example, in the process described herein, the oxidation and reforming reactant streams are passed concurrently in the respective reaction chambers. Generally, the gas streams flow simultaneously; but at certain times such as start up and/or shut down, the reforming side flow may not be reforming reactant flow, but some other gas flow such as N₂, et al.

As used herein, “thermally conductive surface” refers to a surface, typically comprising a metal or metal alloy, between the oxidation and reaction chambers.

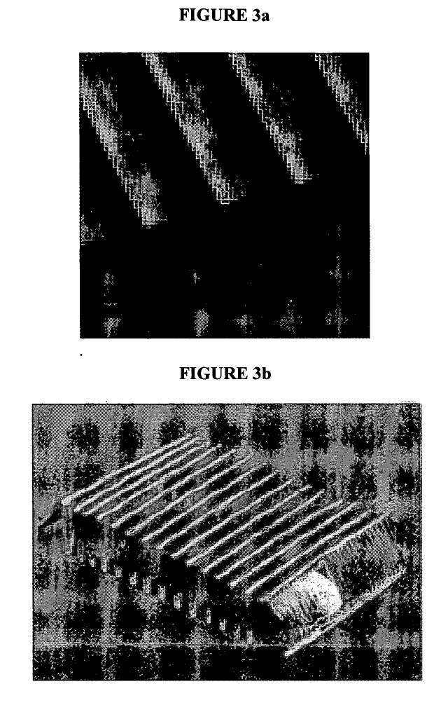

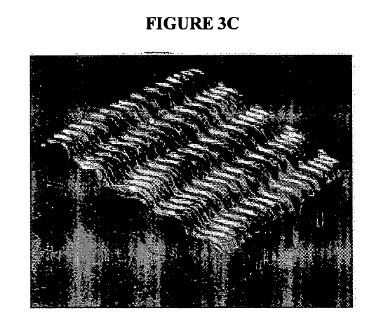

As used herein, “heat exchange fin” refers to a piece of thermally conductive material, typically of metal or metal alloy, that extends in the oxidation or reforming chamber from the thermally conductive surface in the direction normal to the flow of the oxidation and reforming streams. The fin is typically mounted so that its plane is normal to the axis of the oxidation and reforming chambers. However, the fin may instead be mounted to have its plane at an angle with respect to the axis. In certain embodiments, the fin types that can be used include straight fin, perforated fin, offset fin, louvered fin, wave fin and corrugated fin. Exemplary fins are depicted in FIGS. 3a, 3b and 3c.

As used herein, “synthesis gas” or “syngas” refers a mixture that includes hydrogen and carbon monoxide. In addition synthesis gas may comprise water, carbon dioxide, unconverted light hydrocarbon feedstock and various impurities.

As used herein, “at least a part of heat” refers to at least about 10% of the heat generated, at least about 20% of the heat generated, at least about 30% of the heat generated, at least about 40% of the heat generated or at least about 50% of the heat generated.

As used herein, “substantial part of heat” refers to more than 50% of the heat generated, more than about 60% of the heat generated, more than about 70% of the heat generated, more than about 80% of the heat generated, more than about 90% of the heat generated or more than about 95% of the heat generated.

### DESCRIPTION OF EMBODIMENTS

In one embodiment, provided herein is a process and a reactor to generate a synthesis gas via steam reforming of methane. In the synthesis gas generation process provided herein, methane is converted to a synthesis gas comprising carbon monoxide and hydrogen. The process involves concurrently providing an oxidation reactant stream through an oxidation chamber and a reforming reactant stream through a steam reforming chamber, wherein, the oxidation chamber is adjacent to the reforming chamber, and the oxidation chamber and the reforming chamber are separated by a thermally conductive surface.

The oxidation reactant stream used in the process comprises hydrogen, methane, carbon monoxide and oxygen. The oxygen or oxygen source may comprise molecular oxygen, air or other oxidants, such as nitrogen oxides, which can function as a source of oxygen. The oxygen source may be carbon dioxide, carbon monoxide or a peroxide (e.g., hydrogen peroxide). Gaseous mixtures containing oxygen, such as mixtures of oxygen and air, or mixtures of oxygen and an inert gas (e.g., helium, argon, etc.) or a diluent gas (e.g., carbon dioxide, water vapor, etc.) may also be used.

The reforming reactant stream used in the process comprises water and methane. As used herein, the term, “water” generally includes, liquid water, combinations of liquid water and steam, and steam.

Steam methane reforming (“SMR”) comprises an endothermic reaction requiring 205.9 KJ/mol of heat and proceeds according to the following equation:

CH₄+H₂O→CO+3H₂

The process begins by providing an oxidation reactant stream in the oxidation chamber through an inlet and a reforming reactant stream through an inlet in the reforming chamber. The oxidation chamber inlet and the reforming chamber inlet of the reactor are situated adjacent to each other.

In one embodiment, the oxidation reaction is conducted in the presence of an oxidation catalyst. Any oxidation catalyst known to one of skilled in the art could be used. Exemplary oxidation catalysts include, but are not limited to rhodium, iridium, nickel, palladium, platinum, carbide of group VIb and combinations thereof.

In one embodiment, the reforming reaction is conducted in the presence of a steam methane reforming (SMR) catalyst. Any SMR catalyst known to one of skilled in the art could be used. Exemplary SMR catalysts include, but are not limited to rhodium, iridium, nickel, palladium, platinum and combinations thereof.

In certain embodiments, the oxidation reactant stream enters the oxidation chamber inlet at a temperature of about 25° C.-350° C. In certain embodiments, the oxidation reactant stream enters the oxidation chamber inlet at a temperature of at least about 25, at least about 50, at least about 75, at least about 100, at least about 125, at least about 150, at least about 175, at least about 200, at least about 225, at least about 250, at least about 250, at least about 275, at least about 300, at least about 325 or about 350° C. In certain embodiments, the oxidation reactant stream enters the oxidation chamber inlet at a temperature of about 100-250° C. about 130-230° C., about 150-220° C., about 170-210° C. or about 180-210° C.

The pressure in the oxidation chamber is maintained at a range suitable to overcome the total pressure drop to provide the required air flow. In certain embodiments, the pressure in the oxidation chamber is maintained at about 1-5 psig, about 1-3 psig or about 1-2 psig.

In certain embodiments, the flow rate of oxygen in the oxidation chamber is maintained at about 500-1500 or about 700-1200 slpm. In certain embodiments, the flow rate of oxygen in the oxidation chamber is maintained at about 900-1100 slpm.

In certain embodiments, the reforming reactant stream enters the reforming chamber inlet at a temperature of about 400° C.-800° C. In certain embodiments, the reforming reactant stream enters the reforming chamber inlet at a temperature of at least about 400, at least about 430, at least about 450, at least about 500, at least about 550, at least about 600, at least about 650, at least about 700, at least about 750 or about 800° C. In certain embodiments, the reforming reactant stream enters the reforming chamber inlet at a temperature in a range of about 350-500° C., about 370-490° C., about 390-480° C., about 400-470° C. about 410-460° C., about 420-450° C., about 430-450° C. or about 440-460° C.

In certain embodiments, the pressure in the reforming chamber is maintained such that reformed stream has pressure required for the downstream PSA, membrane separator, Fisher-Tropsch reactor or alcohol synthesis reactor while maintaining the CH4 in the SMR at an acceptable conversion. In certain embodiments, the pressure is maintained at about 1 atm to 20 atm or about 5 atm to 15 atm. In certain embodiments, the pressure is maintained at about 7-12 atm or about 10 atm.

In certain embodiments, the flow rate of the reforming reactant stream is maintained at about 0.05-0.3 MMScfd, about 0.08-0.2 MMScfd or about 0.08-0.15 MMScfd for small scale hydrogen application such as hydrogen fuelling station. In certain embodiments, the flow rate of the reforming reactant stream is maintained at about 0.1 MMScfd for small scale hydrogen application such as hydrogen fuelling station. In certain embodiments, the flow rate of the reforming reactant stream is maintained at about 5-20 MMScfd, about 7-15 MMScfd or about 8-12 MMScfd for small scale industrial GTL (Gas-to-Liquid) application. In certain embodiments, the flow rate of the reforming reactant stream is maintained at about 10 MMScfd for small scale industrial GTL (Gas-to-Liquid) application.

In the oxidation chamber, hydrogen, carbon monoxide and methane gas in the oxidation reactant stream oxidize in the presence of the oxidation catalyst. At least a part of the heat generated in the reaction is transferred to the reforming chamber through the heat exchange fins on the thermally conductive wall. The balance of the heat generated increases the temperature of the oxidation reactant stream.

In certain embodiments, the oxidation reactant stream undergoes homogeneous combustion when the temperature of the oxidation reactant stream reaches about 500-600° C., 520-590° C., 530-590° C. or about 540-585° C. The resulting high temperature and a substantial part of the heat are transferred to the reforming chamber thereby improving the steam methane reforming rate. The reforming product stream exits the reforming chamber at a temperature of about 750° C. to about 850° C. or about 770° C. to about 825° C. In one embodiment, the reforming product stream exits the reforming chamber at the temperature of about 780-820° C. or 790-810° C.

In one embodiment, the temperature of the combustion reaction is maintained such that the temperature in the oxidation chamber is below the catalyst sintering temperature. The combustion temperature is maintained by controlling the air flow rate to the oxidation chamber.

The flow rates of the various feed streams, air, methane-containing gas and oxidation feed gas, are controlled by means such as changing blower, pump, and compressor flows, automated or manual control valves, a system controller that automates control over the flows of oxygen to the oxidation chamber, and fuel and water to the reforming chamber, and other similar controllers. Other control means will be apparent to one skilled in the art and are included within the scope of the processes and apparatus described herein.

In one aspect, the process for generating synthesis gas comprises: providing an oxidation reactant stream into an oxidation chamber such that the temperature in the oxidation chamber increases from an inlet of the oxidation chamber to an outlet of the oxidation chamber; concurrently providing a steam methane reforming stream in a reforming chamber that is adjacent to the oxidation chamber, wherein the oxidation chamber and the reforming chamber are separated by a thermally conductive wall; transferring at least a part of the heat generated in the oxidation chamber to the reforming chamber; and reacting the reforming reforming stream to generate the synthesis gas.

In one embodiment, the process further comprises the step of feeding the synthesis gas generated to a pressure swing unit to produce pure hydrogen.

In some embodiments, purification is carried out in a pressure swing adsorption (“PSA”) unit having adsorptive materials that selectively adsorb impurities and allow a hydrogen-enriched reformate to pass. In the PSA unit, by-products (CO and CO₂) and unconverted CH₄ in the process gas are selectively adsorbed and hydrogen is allowed to pass. When the PSA unit is fully saturated with by-products, it can be regenerated using a pressure using technique and a small amount of hydrogen. A mixture of CO, CO₂, CH₄, and hydrogen exiting the PSA unit during regeneration cycles is typically referred to as off-gas. The fuels in the off-gas can be combusted to produce heat that can be used to preheat reactant streams for the steam reforming reaction.

Suitable PSA units include those known in the art for separating hydrogen from a process stream, such as are described in U.S. Pat. No. 4,238,204 issued Dec. 9, 1980 to Perry; U.S. Pat. No. 4,690,695 issued Sep. 1, 1987 to Doshi; U.S. Pat. No. 5,256,174 issued Oct. 26, 1993 to Kai et al.; U.S. Pat. No. 5,435,836 issued Jul. 25, 1995 to Anand et al.; U.S. Pat. No. 5,669,960 issued Sep. 23, 1997 to Couche: U.S. Pat. No. 5,753,010 issued May 19, 1998 to Sircar et al.; and U.S. Pat. No. 6,471,744 issued Oct. 29, 2002 to Hill, the descriptions of which are incorporated herein by reference. In some embodiments, the purification unit will comprise a compact PSA. Suitable compact PSAs can include a rotary-type PSA such as are described in U.S. Pat. No. 6,063,161 issued May 16, 2000 to Keefer et al. and in U.S. Pat. No. 6.406,523 issued Jun. 18, 2002 to Connor et al., the descriptions of which are incorporated herein by reference. Compact PSAs having rotary elements are commercially available from Questair Technologies, Inc. of Burnaby, Canada.

In one embodiment, the process for generating a synthesis gas further comprises the step of feeding the synthesis gas to a membrane separator to produce pure hydrogen. In the membrane separator, the synthesis gas is passed through a hydrogen-separating membrane to selectively recover hydrogen from the synthesis gas. The hydrogen-separating membranes comprise hydrogen-permeable metals, such as palladium and alloys of palladium. Suitable membrane separators those known in the art, such as are described in U.S. Pat. Nos. 5,741,474; 6,767,389; 7,005,113; 7,195,663 and U.S. Application No. 20060248800, the disclosures of which are all incorporated herein by reference.

In certain embodiments, the synthesis gas is fed to a membrane separator to produce synthesis gas with a desired H₂ to CO ratio. In such embodiments, the synthesis gas passes through a hydrogen-separating membrane to separate a predetermined quantity of hydrogen from the synthesis gas to obtain the desired ratio of H₂ to CO in the synthesis gas. In certain embodiment, the desired ratio of hydrogen to carbon monoxide is about 2:1, 2.08:1, 2.2:1 or 2.5:1. In certain embodiments, the ratio of H₂ to CO in the synthesis gas is suitable for feed in the Fischer-Tropsch reactor.

In one embodiment, the process for generating a synthesis gas further comprises feeding the synthesis gas to a Fischer-Tropsch reactor to produce a liquid hydrocarbon. Any Fischer-Tropsch reactor known in the art can be used, such as those described in U.S. Pat. Nos. 5,252,613 and 7,108,835, the disclosures of which are all incorporated herein by reference.

In one embodiment, the process for generating a synthesis gas further comprises feeding the synthesis gas to an alcohol synthesis reactor to produce alcohols including methanol, ethanol and higher alcohols. Any alcohol synthesis reactor known in the art can be used, such as those described in U.S. Pat. Nos. 4,973,453; 6,130,259; 6,939,999, the disclosures of which are all incorporated herein by reference.

Also provided is a reactor for generating a synthesis gas comprising an oxidation chamber and a reforming chamber, wherein the oxidation chamber and the reforming chamber are separated by a thermally conductive surface.

The thermally conductive surface typically comprises a metal, including a metallic alloy. Any metal or alloy that is chemically compatible with the oxidation and reforming reactions is potentially suitable. Potentially suitable metals include, but are not limited to, aluminum, brass, copper, stainless steel, mild steel, titanium, nickel, inconel and chromalloy.

The reforming chamber comprises an inlet for a reforming reactant stream, an outlet for the reforming product stream, and a reforming catalyst disposed on a plurality of heat exchange fins. The oxidation chamber comprises an inlet for an oxidation reactant stream, an outlet for the oxidation product stream, and an oxidation catalyst disposed on a plurality of heat exchange fins. The oxidation and reforming chambers are arranged such that the inlet of the oxidation chamber is adjacent to the inlet of the reforming chamber.

The heat exchange fins used herein can have any geometry suitable for use in the oxidation and reforming chambers. In certain embodiments the heat exchange fins are planar fins (or plate fins), individually attached fins (e.g., a series of circular fins attached at intervals along the length of the thermally conductive surface), or any other type of heat exchange fins known to one skilled in the art. Exemplary heat exchange fins are depicted in FIGS. 3a, 3b and 3c.

The heat exchange fins typically comprise a metal, including a metallic alloy. Any metal or alloy that is suitable for use in the oxidation and reforming reactions is potentially suitable. Exemplary metals include, but are not limited to, aluminum, brass, copper, stainless steel, mild steel, titanium, nickel, inconel and chromalloy. In certain embodiments, the material of the thermally conductive surface the heat exchange fins is the same. In other embodiments, the material of the thermally conductive surface the heat exchange fins is different.

In certain embodiments, the catalysts fins are brazed on the thermally conductive surface. In certain aspects other methods known to one of skill in the art can be used for attaching the fins to the thermally conductive surface, such methods include soldering, welding, extrusion, mechanical fit and tension wound.

In certain embodiments, the oxidation catalyst is deposited on the heat exchange fins. In certain embodiments, the reforming catalyst is deposited on the heat exchange fins.

In one embodiment, the reactor further comprises a pressure swing unit pressure swing unit to produce pure hydrogen downstream from reforming chamber.

In one embodiment, the reactor further comprises a membrane separator downstream from reforming chamber to produce pure hydrogen or to produce synthesis gas with a desired H₂ to CO ratio.

In one embodiment, the reactor further comprises a Fischer-Tropsch reactor downstream from reforming chamber to produce a liquid hydrocarbon.

In one embodiment, the reactor further comprises an alcohol synthesis reactor downstream from reforming chamber to produce alcohols.

In one embodiment, the reactor further comprises a membrane separator and a Fischer-Tropsch reactor downstream from a reforming chamber.

In one embodiment, the reactor further comprises a membrane separator and an alcohol synthesis reactor to produce alcohols downstream from a reforming chamber.

In certain embodiments, the reactor has the shape of plate-fins, wherein there is at least one oxidation chamber and at least one reforming chamber. The oxidation chamber comprises an inlet for oxidation reactant stream and the reforming chamber comprises an inlet for the reforming reactant stream.

In one aspect, the reactor comprises multiple oxidation and reforming chambers. The oxidation and reforming chamber are arranged in an alternate fashion such that the oxidation and reforming streams can be provided in alternate chambers.

In one aspect, the reactor is cylindrical and comprises an outer cylindrical chamber and inner cylindrical chamber. In one embodiment, the outer cylindrical chamber comprises the oxidation, chamber and the inner cylindrical chamber comprises the reforming chamber. In another embodiment, the outer cylindrical chamber comprises the reforming chamber and the inner cylindrical chamber comprises the oxidation chamber.

The process and the reactor provided herein reduce the cost associated with higher air power consumption because the air flow required in the process is often significantly lower than that required in conventional counter-current steam methane reform reactions. The reactor design having the oxidation and reforming chambers adjacent to each other often may reduce heat loss in transferring heat from the exothermic oxidation reaction to the endothermic reforming reaction. The higher temperatures at the outlets of the reforming and oxidation chamber often may result in high methane conversion rates.

Although only exemplary embodiments are specifically illustrated and described herein, it will be appreciated that many modifications and variations of the process and apparatus described herein, are possible in light of the above teachings and within the purview of the appended claims without departing from the spirit and intended scope of the claimed subject matter.

All publications and patent applications mentioned In this specification are herein incorporated by reference to the same extent as if each individual publication or patent application, was specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A process for generating a synthesis gas comprising: concurrently providing an oxidation reactant stream through an oxidation chamber and a reforming reactant stream through a steam reforming chamber,
a) wherein the oxidation chamber is adjacent to the reforming chamber and the oxidation chamber and the reforming chamber are separated by a thermally conductive surface;
b) wherein the reforming chamber comprises: an inlet for a reforming stream, an outlet for the reforming stream, and a reforming catalyst disposed on a plurality of heat exchange fins;
c) wherein the oxidation chamber comprises: an inlet for an oxidation reactant stream, an outlet for the oxidation reactant stream, and an oxidation catalyst disposed on a plurality of heat exchange fins;
d) wherein the inlet of the oxidation chamber is adjacent to the inlet of the reforming chamber; and
e) wherein the plurality of heat exchange fins are brazed on the thermally conductive surface.

2. The process of claim 1, wherein the oxidation reactant stream comprises hydrogen, methane, carbon monoxide and oxygen.

3. The process of claim 2, wherein the oxidation reactant stream enters the oxidation chamber at a temperature of from about 25° C. to about 350° C.

4. The process of claim 3, wherein the oxidation reactant stream enters the oxidation chamber at the temperature of from about 180 to about 210° C.

5. The process of claim 4, wherein the temperature at the outlet of the oxidation chamber is from about 450° C. to about 900° C.

6. The process of claim 5, wherein the temperature at the outlet of the oxidation chamber is from about 540 to about 560° C.

7. The process of claim 1, wherein the oxidation catalyst comprises palladium, platinum, copper or a combination thereof.

8. The process of claim 1, wherein the reforming reactant stream comprises water and methane.

9. The process of claim 1, wherein the steam reforming catalyst comprises platinum, palladium, rhodium, ruthenium, iridium, nickel or a combination thereof.

10. The process of claim 1, wherein the reforming reactant stream enters the reforming chamber at a temperature of from about 400° C. to about 800° C.

11. The process of claim 10, wherein the reforming reactant stream enters the reforming chamber at the temperature of from about 430 to about 460° C.

12. The process of claim 1, wherein the temperature at the outlet of the reforming chamber is from about 750° C. to about 850° C.

13. The process of claim 12, wherein the temperature at the outlet of the reforming chamber is from about 780 to about 810° C.

14. The process of claim 4, further comprising oxidizing the oxidation reactant stream to generate heat.

15. The process of claim 14, wherein at least a part of the heat generated in the oxidation chamber is transferred to the reforming chamber.

16. The process of claim 14, wherein at least a part of the heat generated in the oxidation chamber is transferred to the oxidation reactant stream thereby increasing the temperature of the oxidation reactant stream to from about 500 to about 600° C.

17. The process of claim 16, wherein the temperature of the oxidation reactant stream is from about 540 about 585° C.

18. The process of claim 17 further comprising homogeneously combusting the oxidation reactant stream.

19. The process of claim 18, wherein a substantial part of the heat generated in the combustion is transferred to the reforming chamber.

20. The process of claim 19, wherein the oxidation chamber further comprises a combustion catalyst.

21. A process for generating a synthesis gas, comprising:
a) providing an oxidation reactant stream into an oxidation chamber such that the temperature in the oxidation chamber increases from an inlet of the oxidation chamber to an outlet of the oxidation chamber due to a heat generated in an oxidation reaction;
b) concurrently providing a steam methane reforming stream in a reforming chamber, wherein the oxidation chamber and the reforming chamber are separated by a thermally conductive wall;
c) transferring at least a part of the heat generated in the oxidation chamber to the reforming chamber; and
d) reacting the reforming stream to generate the synthesis gas.

22. The process of claim 21, wherein at least a part of the heat generated in the oxidation chamber is transferred to the oxidation reactant stream.

23. The process of claim 22 further comprising combustion of the oxidation reactant stream.

24. The process of claim 1 further comprising feeding the synthesis gas generated to a pressure swing unit to produce pure hydrogen.

25. The process of claim 1 further comprising feeding the synthesis gas generated to a membrane separator to produce pure hydrogen.

26. The process of claim 1 further comprising feeding the synthesis gas generated to a membrane separator to produce the synthesis gas with a desired H₂/CO ratio.

27. The process of claim 1 further comprising feeding the synthesis gas generated to a Fischer-Tropsch reactor to produce liquid hydrocarbons.

28. The process of claim 1 further comprising feeding the synthesis gas generated to an alcohol synthesis reactor to produce alcohols.

29. The process of claim 26, further comprising feeding the synthesis gas to a Fischer-Tropsch reactor to produce liquid hydrocarbons.

30. The process of claim 26, further comprising feeding the synthesis gas to an alcohol synthesis reactor to produce an alcohol.

31. A reactor for generating a synthesis gas comprising an oxidation chamber and a reforming chamber, wherein
a) the oxidation chamber and the reforming chamber are separated by a thermally conductive surface;
b) the reforming chamber comprises: an inlet for a reforming stream, an outlet for the reforming stream, and a reforming catalyst disposed on a plurality of heat exchange fins;
c) the oxidation chamber comprises: an inlet for an oxidation reactant stream, an outlet for the oxidation product stream, and an oxidation catalyst disposed on a plurality of heat exchange fins,
d) the inlet of the oxidation chamber is adjacent to the inlet of the reforming chamber; and
e) the heat exchange fins are brazed on the thermally conductive surface.

32. The reactor of claim 31, wherein the oxidation catalyst comprises palladium, platinum, copper or a combination thereof.

33. The reactor of claim 31, wherein the reforming catalyst comprises platinum, palladium, rhodium, ruthenium, iridium, nickel or a combination thereof.

34. The reactor of claim 31 further comprising a pressure swing adsorption unit downstream of the reforming chamber.

35. The reactor of claim 31 further comprising a membrane separator downstream of the reforming chamber.

36. The reactor of claim 31 further comprising a Fischer-Tropsch reactor downstream of the reforming chamber.

37. The reactor of claim 31 further comprising an alcohol synthesis reactor downstream of the reforming chamber.

38. The reactor of claim 35 further comprising a Fischer-Tropsch reactor downstream of the membrane separator.

39. The reactor of claim 35 further comprising an alcohol synthesis reactor downstream of the membrane separator.

40. The reactor of claim 31 further comprising multiple oxidation and reforming chambers arranged such that each oxidation chamber alternates with a reforming chamber.

41. A cylindrical reactor for generating a synthesis gas comprising an outer cylindrical chamber and an inner chamber, wherein
a) the outer chamber and the inner chamber are separated by a thermally conductive surface;
b) the inner chamber comprises: an inlet for a reforming stream, an outlet for the reforming stream, and a reforming catalyst disposed on a plurality of heat, exchange fins;
c) the outer chamber comprises: an inlet for an oxidation reactant stream, an outlet for the oxidation product stream, and an oxidation catalyst disposed on a plurality of heat exchange fins,
d) the inlet of the outer chamber is adjacent to the inlet of the inner chamber; and
e) the heat exchange fins are brazed on the thermally conductive surface.
